(19) United States
(12) Patent Application Publication
(10) Pub. No.: US 2021/0137408 A1
(43) Pub. Date: May 13, 2021

(54) SYSTEM AND METHOD FOR CONDUCTIVITY-BASED IMAGING

(71) Applicant: NAVIX INTERNATIONAL LIMITED, Tortola (VG)

(72) Inventors: Shlomo BEN-HAIM, Milan (IT); Michael WILK, Haifa (IL); Zalman IBRAGIMOV, Rehovot (IL)

(73) Assignee: NAVIX INTERNATIONAL LIMITED, Tortola (VG)

(21) Appl. No.: 17/257,157

(22) PCT Filed: Jul. 4, 2019
(86) PCT No.: PCT/EP2019/068014

§ 371 Date: Dec. 30, 2020

Related U.S. Application Data
(60) Provisional application No. 62/693,921, filed on Jul. 4, 2018.

(51) Int. Cl. A61B 5/0536; A61B 5/0538; G16H 30/40; G16H 50/20

(52) U.S. Cl. CPC A61B 5/0536; A61B 5/0538; A61B 5/0044; G16H 50/20; G16H 30/40

(57) ABSTRACT

A method of performing conductivity-based imaging is disclosed comprising exciting at least one pair of electrodes according to an excitation scheme. The at least one pair of electrodes comprising a surface electrode located on the surface of an examined body and at least one in-body electrode located inside of the examined living body. Voltages developing on the surface electrodes and on the in-body electrodes during the excitation according to the excitation scheme arc measured, an inverse problem is solved to achieve a 3D conductivity map from the measured voltages, and a 3D image of the body tissues based on the 3D conductivity map is provided.

### FIELD AND BACKGROUND OF THE INVENTION

The present invention, in some embodiments thereof, relates to constructing a conductivity map and medical imaging and, more specifically, but not exclusively, to systems and methods for conductivity-based imaging, e.g., for reconstruction of body tissues and organs.

Electrical Impedance Tomography (EIT) system and method of medical imaging, as is known in the art, is implemented by deploying electrodes at the body's surface of a subject, injecting electrical excitation to some of the employed electrodes, measuring the electrical signals received at the other employed electrodes, calculating, based on the measured signals, 3D image(s) of tissues and organs inside the body and providing display of the calculated 3D images.

The 3D imaging is based on the fact that muscle and blood conduct the applied currents better than fat, bone, or lung tissue. However, the 3D imaging that is based on reconstructed conductivity has found only limited utility, because of the low resolution of the obtained images.

There is a need for system and method that provide accurate imaging of body organs and lumens, which do not employ ionized radiation or which minimize ionized radiation.

### SUMMARY OF THE INVENTION

The present invention, in some embodiments thereof, relates to constructing a conductivity map and medical imaging and, more specifically, but not exclusively, to systems and methods for conductivity-based imaging, e.g., for reconstruction of body tissues and organs.

A method of performing conductivity-based imaging is disclosed comprising exciting at least one pair of electrodes according to an excitation scheme, the at least one pair of electrodes comprising a surface electrode located on the surface of an examined living body and at least one in-body electrode (also referred herein below intra-body electrode) located inside of the examined living body, measuring and recording voltages developing on the surface electrodes and on the in-body electrodes during the excitation according to the excitation scheme, solving an inverse problem to achieve a 3D conductivity map from the recorded voltages and optionally providing a 3D image of the body tissues based on the 3D conductivity map. For example, a 3D conductivity may provide a three-dimensional spatial distribution of conductivity values associating values of conductivity of a material, here body tissue, with a corresponding voxel or discrete volume in space where the material is located.

A method of providing 3D conductivity map of body tissues is disclosed comprising exciting at least one pair of electrodes according to an excitation scheme, the at least one pair of electrodes comprising a surface electrode located on the surface of an examined living body and at least one in-body electrode located inside of the examined living body, measuring voltages developing on the surface electrodes and on the in-body electrodes during the excitation according to the excitation scheme, solving an inverse problem to obtain a 3D conductivity map from the measured voltages. The method may comprise providing (e.g., displaying) a 3D image of the body tissues based on the 3D conductivity map.

In some embodiments the excitation is applied to at least one additional pair of electrodes that comprises a surface electrode located on the surface of an examined body and at least one in-body electrode located inside of the examined living body.

In some embodiments the steps of exciting and measuring are repeated M times, while the in-body electrodes are moved between cycles of excitation.

In some embodiments a step of averaging and weighing the measurements of the step of measuring is performed before the step of solving.

A method of imaging body tissues is disclosed comprising exciting at least one pair of electrodes according to an excitation scheme, the at least one pair of electrodes comprising a surface electrode located on the surface of an examined living body and at least one in-body electrode located inside of the examined living body, measuring voltages developing on the surface electrodes and on the in-body electrodes during the excitation according to the excitation scheme, solving an inverse problem to obtain a 3D conductivity map from the measured voltages. The method may comprise providing (e.g., displaying) a 3D image of the body tissues based on the 3D conductivity map.

A method of imaging a volume in an examined living body is disclosed comprising exciting at least one pair of electrodes according to an excitation scheme, the at least one pair of electrodes comprising a surface electrode located on the surface of an examined living body and at least one in-body electrode located inside of the examined living body, measuring voltages developing on the surface electrodes and on the in-body electrodes during the excitation according to the excitation scheme, solving an inverse problem to obtain a 3D conductivity map from the measured voltages. The method may comprise providing (e.g., displaying) a 3D image of the volume based on the 3D conductivity map.

According to some embodiments of the present invention there is provided a method of performing conductivity-based imaging, the method comprising: exciting at least one pair of electrodes according to an excitation scheme, the at least one pair of electrodes comprising a surface electrode located on the surface of an examined body and at least one in-body electrode located inside of the examined body; measuring and recording voltages developing on the surface electrode and on the in-body electrode during the excitation according to the excitation scheme; solving an inverse problem to achieve a 3D conductivity map from the recorded voltages; and providing a 3D image of the body tissues based on the 3D conductivity map.

According to some embodiments, the excitation is applied to at least one additional pair of electrodes that comprises a surface electrode located on the surface of the examined body and at least one in-body electrode located inside of the examined body.

According to some embodiments, the excitation is applied to at least one additional pair of electrodes that comprises two in-body electrodes.

According to some embodiments, the excitation is applied to at least one additional pair of electrodes that comprises two surface electrodes.

According to some embodiments, the steps of exciting and measuring are repeated a defined number of times (M) with the in-body electrodes at different locations inside the body, wherein M is at least two.

According to some embodiments, the steps of exciting and measuring are repeated at a rate of between 10 and 500 times per second.

According to some embodiments, the method further comprising a step of combining measurements obtained when the in-body electrodes were at different locations to a single set of measurements, and wherein the inverse problem is solved for that single set of measurements.

According to some embodiments, the different locations include at least two locations, each in the vicinity of a different structural feature to be imaged within a volume of the examined body.

According to some embodiments, the solving is executed for each of the M measurements separately, and the obtained solutions are averaged to provide the 3D image.

According to some embodiments of the present invention there is provided a method of providing a 3D image of the body tissues. The method comprising: exciting at least one pair of electrodes according to an excitation scheme, the at least one pair of electrodes comprising a surface electrode located on the surface of an examined body and at least one in-body electrode located inside of the examined body; measuring voltages developing on the surface electrode and on the in-body electrode during the excitation according to the excitation scheme; solving an inverse problem to achieve a 3D conductivity map from the measured voltages; and providing a 3D image of the body tissues based on the 3D conductivity map.

According to some embodiments, the excitation is applied to at least one additional pair of electrodes that comprises a surface electrode located on the surface of an examined body and at least one in-body electrode located inside of the examined body.

According to some embodiments, the excitation is applied to at least one additional pair of electrodes that comprises two in-body electrodes.

According to some embodiments, the excitation is applied to at least one additional pair of electrodes that comprises two surface electrodes.

According to some embodiments, the steps of exciting and measuring are repeated a defined number of times (M) with the in-body electrodes at different locations inside the body, wherein M is at least two.

According to some embodiments of the present invention there is provided a method of obtaining a 3D conductivity map of body tissues. The method comprising: exciting at least one pair of electrodes according to an excitation scheme, the at least one pair of electrodes comprising a surface electrode located on the surface of an examined body and at least one in-body electrode located inside of the examined body; measuring voltages developing on the surface electrode and on the in-body electrode during the excitation according to the excitation scheme; and solving an inverse problem to obtain a 3D conductivity map from the measured voltages.

According to some embodiments of the present invention there is provided a method of performing conductivity-based imaging. The method comprising: receiving voltage measurements developed on a surface electrode located on the surface of an examined body and on an in-body electrode located inside of the examined body during an excitation according to an excitation scheme; wherein the excitation scheme includes exciting the surface electrode and the in-body electrode; solving an inverse problem to achieve a 3D conductivity map from the measured voltages; and providing a 3D image of the body tissues based on the 3D conductivity map.

According to some embodiments of the present invention, a method of obtaining a 3D conductivity map of body tissues comprises receiving measured voltages measured on receiving electrodes in response to currents injected to transmitting electrodes, the transmitting and receiving electrodes comprising at least one surface electrode located on the surface of an examined body and at least one in-body electrode located inside of the examined body. For example, at least one of the receiving electrodes may be a surface electrode or at least one of the receiving electrodes may be an in-body electrode. At least one of the transmitting electrodes may be a surface electrode or at least one of the transmitting electrodes may be an in-body electrode. Thus, the measured voltages may comprise one or more of: a voltage measured on a surface electrode in response to current injected to an in-body electrode; a voltage measured on an in-body electrode in response to current injected to an in-body electrode; a voltage measured on an in-body electrode in response to current injected to a surface electrode. The method further comprises solving an inverse problem to obtain a 3D conductivity map from the received voltages.

In some specific examples, each transmitting electrode has current injected to it and transmits at a respective time and frequency, so that the transmissions from each transmitting electrode can be separated from transmissions of other transmitting electrodes. A resulting voltage may be measured at each time and frequency at which a transmitting electrode transmits by one or more receiving electrodes, so that for each transmitting electrode there is one or more pairs of electrodes including that transmitting electrode for the given time and frequency and a respective one of the receiving electrodes for that time and frequency. These pairs may comprise two in-body electrodes or one surface and one in-body electrode (respectively transmitting and receiving, or vice versa). Each pair of electrodes and the corresponding voltage measurement provide a data point that can then be used to solve the inverse problem of finding a conductivity map from the voltage measurements. In some specific cases, all transmitting electrodes may transmit at the same time and at different frequencies, at the same frequency but at different times or a combination of the two. It will be understood that any one transmitting electrode may be a transmitting electrode for a given frequency or frequencies and a receiving electrode at another frequency or frequencies, and/or may be a transmitting electrode for a given time slot or slots and a receiving electrode at another slot or slots. Any one receiving electrode may be a receiving electrode for a given frequency or frequencies and a transmitting electrode at another frequency or frequencies, and/or may be a receiving electrode for a given time slot or slots and a transmitting electrode at another slot or slots. One or more of the transmitting electrodes may be each associated with one or more other transmitting electrodes, for example in one or more pairs, triodes, quartets, etc. of transmitting electrodes and have current injected at the same frequency as its associated other transmitting electrodes, with a defined phase relationship between the respective currents, for example in a pair of transmitting electrodes having current injected at the same frequency and opposite phases, or in a triode of transmitting electrodes having current injected at a common frequency and different phases, for example, 0 degrees, 120 degrees and 240 degrees respectively.

In some embodiments, the measured voltages were measured a defined number of times M with the in-body electrode or electrodes at different locations inside the body each time, wherein M is at least two. In some embodiments, the measured voltages were measured at a rate of between 10 and 500 times per second. These embodiments may comprise a step of combining measurements obtained when the in-body electrode or electrodes were at different locations in a single set of measurements, and solving the inverse problem for that single set of measurements. The different locations may include at least two locations, each in the vicinity of a different structural feature to be imaged within a volume of the examined body. In some cases, the solving may be executed for each of the M measurements separately, and the obtained solutions may be combined, for example, averaged to provide the conductivity map.

In some embodiments, receiving the measured voltages comprises receiving sets of measured voltages, wherein, for each set, respective measured voltages were measured on one or more of the receiving electrodes while injecting respective currents on one or more of the transmitting electrodes. In some embodiments, a measured voltage was measured on a selected electrode when obtaining one set of measurements and a current was injected to the selected electrode when obtaining a second set of measurements. In other words, a given electrode may act as a receiving electrode for one set and as a transmitting electrode for another set.

According to some embodiments of the present invention there is provided a system for performing conductivity-based imaging comprising a controller configured to perform the methods described above when executing executable code stored in its memory.

According to some embodiments of the present invention there is provided a system for performing conductivity-based imaging. The system comprises: a control unit; surface electrodes unit, comprising at least 2 electrodes; intra-body electrodes, comprising at least 2 electrodes; a first communication channel to provide communication between the control unit and the surface electrodes unit; and a second communication channel to provide communication between the control unit and the intra-body electrodes unit.

In any of the above embodiments, the in-body electrodes may be disposed on one or more catheters. Other kinds of in-body electrodes include electrodes disposed on a sheath, guidewire, or any other medical implement that may carry electrodes. In the following, catheter electrodes may always be used as examples of in-body electrodes.

Although the present disclosure relates to embodiments related to conductivity-based imaging and conductance maps, it will be appreciated that it is more generally applicable to dielectric imaging or maps of dielectric properties other than conductivity and conductance, for example admittivity or admittance, impedance, permittivity, and the like.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically depicts deployment of a set of electrodes on and in a body, according to embodiments of the present invention;

FIG. 2 is a schematic illustration of catheter 208, according to embodiments of the present invention;

FIG. 3 schematically depicts electrical field generator/measurer, according to embodiments of the present invention;

FIG. 4 is a schematic block diagram of a system for conductivity-based imaging, according to embodiments of the present invention;

FIG. 5 is a top-level flow of a process for converting a collection of measured voltages on a set of electrodes into a 3D image, according to embodiments of the present invention;

FIG. 6A is a flow chart depicting method for conductivity-based imaging according to embodiments of the present invention;

FIG. 6B is a flow chart depicting method for performing multiple cycles of measurements, according to the invention;

FIG. 7 is a flow chart depicting a method of performing conductivity-based imaging according to some embodiments of the present invention;

FIG. 8A is simulation results of 3D conductivity values reconstructed from voltage measurements made by surface electrodes and in-body electrodes according to some embodiments of the invention; and

FIG. 8B shows the actual structure the reconstruction of which is depicted in FIG. 8A.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

The present invention, in some embodiments thereof, relates to medical imaging and, more specifically, but not exclusively, to systems and methods for conductivity-based imaging, e.g., for reconstruction of body tissues and organs.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. The terms ‘injecting signal’, ‘injecting current’, ‘exciting signal’ and ‘exciting current’ will be all used herein after to describe signals provided to electrodes used in the process of imaging as described below.

The present invention may be a system, a method, and/or a computer program product. The computer program product may include a computer readable storage medium (or media) having computer readable program instructions thereon for causing a processor to carry out aspects of the present invention.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the following detailed description, the terms catheter may refer to any physical carrier of one or more electrodes for insertion the one or more electrodes into a living body—for example: endoscope, colonoscope, enteral feeding tube, stent, graft, etc.

Systems and methods for intra-body-electrode aided conductivity-based imaging may employ one or more surface electrodes deployed on the surface of an examined body preferably around the examined body organ and one or more intra-body electrodes. According to embodiments of the present invention, one or more electrodes may be inserted into a living body, preferably but not solely, carried by or being part of an existing medical device, such as catheter, endoscope, colonoscope, enteral feeding tube, stent, graft, etc. According to some embodiments the insertion of electrodes may be made as part of, or in addition to another medical procedure, e.g. for saving the patient the inconvenience involved in undergoing the physical process more than once. In such instances, according to some embodiments electrical signals that need to be injected to some of the electrodes, and the respective signals that need to be measured on other electrodes of the system, may take advantage of another medical procedure that involves injection and measuring of signals between electrodes (e.g. where such signaling is employed for providing location information of the catheter or other intra-body device used as an electrode carrier) in the body, and may use the signaling process (injection and measuring) for the purpose of intra-body-electrode aided conductivity-based imaging, as is explained in detail below. For example steps 602 and 604 of FIG. 6A or steps 654 and 656 of FIG. 6B or steps 704 and 706 of FIG. 7 may be part of a medical procedures.

Exemplary medical procedures may include catheterization procedures, e.g., cardiac ablation, intestinal ablation, stent or graft deployment, etc. In some embodiments, intra-body-electrode aided conductivity-based imaging may take advantage of the catheter which is already inserted into the living body and the surface electrode provided as part of such medical procedures. For example, imaging of organs (other organs or in addition to the heart—for example: the aorta and/or the esophagus or the entire chest environment) may be performed using intra-body-electrode aided conductivity-based imaging to a patient undergoing a cardiac ablation procedure.

In some embodiments, intra-body-electrode aided conductivity-based imaging may be implemented to existing system(s) by programming such system to use information already received by such system (e.g., for navigation) to obtain imaging of an organ being treated by such system or other organs. For example, in cardiac ablation procedures: catheter(s) carrying one or more intra-body electrodes and surface electrodes may be used in existing procedures to map the inside of a heart chamber, and the intra-body-electrode aided conductivity-based imaging may supply imaging of the entire chest environment, for example, to show the aorta and/or the esophagus.

Damaging the esophagus is one of the more common complications of left atrium ablation, because the esophagus neighbors the left atrium, and is usually dynamic. Esophagus imaging using intra-body-electrode aided conductivity-based imaging may be used in such ablation procedures, optionally taking advantage of information (e.g., signals) already obtained in such procedures.

The following detailed description is in reference to voltage measurements, it should be noted that embodiments of the present invention are not limited to voltage measurements and may deploy other measurements, such as impedance measurements.

Reference is made to FIG. 1, which schematically depicts deployment of a set of electrodes 100 on and in a body, according to embodiments of the present invention. In this example, three pairs of surface electrodes (or surface pads) are shown: 102A/102B, 104A/104B and 106A/106B. The pairs of surface electrodes may be disposed on the body substantially at antipode locations. In some embodiments, a smaller or larger number of surface electrodes may be used, and their number may be even or odd. Additionally, set of electrodes 100 comprises intra-body electrodes 103. In the depicted embodiment, the intra-body electrodes are comprised in catheter 108. Catheter 108 may be insertable into a patient's body. In some embodiments, the intra-body electrodes may be carried by more than one catheter, for examples, two electrode-carrying catheters may be inserted into the patient's body, and used for generating an image as described below.

Surface electrodes 102A/102B, 104A/104B and 106A/106B may be connected to signal source(s) that is/are adapted to inject (or excite) electrical signals in desired strength, frequency and phase. In some embodiments the signal source may be tuned to excite each of the pairs of surface electrodes with signals having opposite phases (or at least substantially well de-phased from each other), for example in order to imitate three substantially spatially orthogonal axes (e.g. X, Y, and Z axes). This is so because such pairwise transmission may be used for locating a catheter, such as catheter 108, inside the body, e.g., for navigation purposes. In addition, voltages developing on the surface electrodes during the excitation of at least one of the intra-body electrodes may be measured and used (together with the known injected currents) for reconstructing a distribution of conductivity (or resistivity) in the volume defined by the body-surface electrodes, e.g., 3D conductivity map. This conductivity distribution may then be used for producing a 3D image of said volume.

Voltages developing on the surface electrodes and/or the intra-body electrodes during the excitation may be measured when the intra-body electrodes are actively moved (e.g., by a physician during a medical procedure) around a region of interest (or inside it or along it, etc.)—e.g., around or inside a tissue to be imaged. In some cases, there may be several regions of interest, and the intra-body electrodes may be “dragged” from one to another, back and forth. For example, inside a left atrium there are many structural features that may be of interest, e.g., the openings of the pulmonary veins (which are of high interest for treating atrial fibrillation), the left atrial appendage, the mitral valve, etc. The catheter may be guided to visit all of them (and especially those relevant to a current treatment), and so the image quality at these regions and their vicinity may be improved.

Reference is now made also to FIG. 2, which is a schematic illustration of catheter 208, according to embodiments of the present invention. Catheter 208 may be, in some embodiments, identical or substantially identical to catheter 108 of FIG. 1. Catheter 208 may comprise one or more electrodes (also referred to herein as intra-body electrodes or in-body electrodes), and in the drawn example four electrodes 210, 212, 214, 216. Each of the electrodes may have connection wire 220, 226, 224, 222, respectively, to enable connecting to electrical excitation unit, such as electrical field generator/measurer, e.g. as described with respect to FIG. 3 hereinafter. Electrodes 210, 212, 214, 216 may be disposed spaced from each other along the longitudinal axis of catheter 208 by longitudinal distances 211, 213, 215. The longitudinal distances may be, for example, in the range of lower than 1 millimeter or few millimeters and up to 1-2 cm or up to 4-6 cm between the farthest intra-body electrodes. In some embodiments it may be beneficial to have the electrodes spaced apart by a distance that is in the magnitude of order of the size of the scanned organ, or less.

In embodiments of the present invention schemes of electrical excitations of surface electrodes and/or intra-body electrodes(also referred herein as excitation scheme or scheme of excitation) yield voltages measurable on one or more of the electrodes. The intra-body electrodes can be catheter electrodes. The voltage readings (voltages measured on one or more surface electrodes and/or intra-body (e.g., catheter) electrodes) may be used to reconstruct a spatial distribution of the electrical conductivity of tissues through which the electrical signals pass (may be referred to herein as 3D conductivity map). Schemes of excitation may comprise selection of the transmitting electrode(s), selection of the frequency of the transmitted signals, selection of the amplitude of each of the transmitted signals, selected duration of the transmission, selection phase differences (or de-phasing) between signals transmitted concurrently from two or more electrodes at a same frequency, and the like. It will be noted that excitation schemes may comprise sets of signal frequencies (transmission frequencies) that may be selected to support one or more needs such as operating in different frequencies to cover different transmissivities of the body tissues along a certain signal path, thereby collecting more information of the tissue's shape. In another example, transmission frequencies may be selected to enable good separation between the transmitted and the received signal, or good separation between signals transmitted concurrently from different electrodes. While separating between signals transmitted concurrently from different electrodes may be achieved with signals separated from each other even in a few kHz, covering different transmissivities may benefit from large frequency differences, for example, frequencies spanning the frequency range between 10 kHz and 100 KHz.

Transmitted signals may be transmitted from one or more of the electrodes, and voltages developing on one or more of the electrodes during the excitation may be received and recorded for further processing. Preferably, voltages developing on all the electrodes are recorded, or voltages developing on all but the transmitting electrodes are recorded. The voltages may be indicative of the conductivity of body tissues through which the signal passed. Since the conductivity along any electrical path of a signal is indicative of the nature of the tissue along that path, the more different signal paths are sampled, the richer is the data on the nature of the tissues, and a more accurate image (e.g., of higher resolution) may be produced from that data. Accordingly, excitation schemes may be used to invoke transmission from, for example, at least one of the intra-body (e.g., catheter) electrodes and the resulting voltages developing on at least all of the surface electrodes may be recorded, thereby providing, in the example of FIG. 1, indication of six different conductivities, which are indicative of the conductivity of the body tissues along six respective signal paths. The paths along which transmitted signals pass are not known, as the signals do not travel in straight lines, but mainly along paths of minimal resistivity. Yet, the large number of measurements of spatial conductivity values, which may represent, for a large number of points in the examined body organ, measurements of more than one signal path that passes through a certain point, enables reconstructing a detailed 3D map of conductivity values, which may be translated to a 3D image of the imaged tissue (e.g., of the organ).

In some embodiments, excitation schemes may be used to invoke transmission from at least one of the intra-body (e.g., catheter) electrodes and the resulting voltages developing on all of the intra-body electrodes may be recorded, or the resulting voltages developing on all of the intra-body electrodes not transmitting may be recorded, thereby providing, in the example of FIG. 1, indication of four different signal paths, which are indicative of the conductivity of the body tissues along the respective paths.

Additionally, one or more transmitted signals may be transmitted from at least one of the surface electrodes and the resulting voltages developing on the other surface electrodes may be measured and recorded, thereby providing conductivity information related to signal paths through body tissues extending between the transmitting surface electrode and the at least one receiving surface electrode, which may provide indication of the tissues of the body closer to the body surface.

In some embodiments, at least some of the excitations may be by electrode pairs, transmitting simultaneously at the same frequency and in opposite phases. In some embodiments, such electrode pair may consist of two surface electrodes or two intra-body electrode electrodes. In some embodiments, such an electrode pair may consist of one intra-body electrode and one surface electrode.

In some embodiments, at least some of the excitations may be by electrode groups of three or more electrodes, transmitting simultaneously at the same frequency and in controlled phase relations between them. In some embodiments, each such electrode group may consist of intra-body electrodes or surface electrodes. In some embodiments, one or more of the groups may include both an intra-body electrode and a surface electrode.

In embodiments where the surface electrodes are also used for navigation, the surface electrodes may transmit in pairs (each pair transmitting at a common frequency, and in two opposite phases), and the intra-body electrodes may each transmit at a different frequency. In some such embodiments, voltages are read only on the intra-body electrodes, and in some embodiments, voltages are also read on the surface electrodes.

As mentioned above, processing of the measured voltages on the various electrodes may be used, additionally to the creation of database of 3D measurements (from which a 3D conductivity map may be produced, as is explained below), also for tracking and positioning the catheter inside the body. Tracking and positioning of the catheter inside the body may be used for medical procedures.

Adding intra-body electrodes, such as catheter electrodes, located inside the body to imaging systems (such as EIT systems) that use only body surface electrodes provides imaging data of much higher quality (e.g., of high definition), in comparison to what is achieved with imaging using only surface electrodes, at least with the same number of electrodes. The improved quality may be proved by comparing its resulting images to images of a known phantom of the body organ, directly measuring the conductivity at some points, and comparing the imaged values (i.e., the conductivity values obtained from the voltage readings) to the directly measured values. Alternatively to measuring a phantom, the quality may be evaluated by creating synthetic data (e.g., electromagnetic simulation), and comparing the imaging results to the simulation.

The plurality of voltage measurements v_{(i,j)} between pairs i, j of electrodes, performed as described above, when a plurality of different excitations is applied over time to a plurality of electrodes and measured by a plurality of electrodes, creates a collection V_{(i,j)} of voltage measurements. For example, for a pair of electrodes i,j, one electrode i of the pair transmits at a certain time and frequency (in response to current injection to electrode i) and the resulting voltage v(i,j) is measured at that time and frequency at the other electrode j of the pair. The collection V_{(i,j)} of voltage measurements may be obtained when the intra-body electrodes are located at different positions within the body (e.g., as the catheter moves inside an organ).

The collection of voltage measurements may be converted to a collection of spatial conductivity values σ_{(x,y,z)}, assigning a calculated conductivity value to points in a defined 3D volume. The points σ_{(x,y,z)}, with their assigned conductivity values may be included in a large collection (or a cloud) of spatial values, hereinafter denoted R.

It will be appreciated that the body volume that may be imaged according to embodiments of the present invention may be defined as a body volume confined between/among a set of surface electrodes usable in the imaging process.

In practice, the intra-body electrodes are typically catheter electrodes, so they may move with the catheter inside the body, when the catheter is moved, e.g. along a body lumen or inside a heart chamber or other organ(s). Solving the 3D conductivity map (i.e. calculating the conductivity value for the collection of 3D points in the scanned volume of the body based on voltages measured at the surface of the imaged volume and inside it or around it) may not require knowledge of the position of the electrodes, (other than knowing which are at the surface and which are inside the body), but the solution depends on that location.

Consider a catheter with a single electrode. At each position of the catheter, the readings at the catheter electrode (and at the surface electrodes, in the frequency transmitted by the catheter electrode) are different, and so are the reconstructions obtained from them. These different reconstructions may be combined, for example, by assigning to each region a conductivity value, equal to an average of the conductivity values assigned to that region in each of the different reconstructions. As the individual reconstructions may vary in quality, the averaging may be weighted by the quality.

Additionally or alternatively, different readings from different places may be combined to provide a single reconstruction. Excitations excited at different times don't interact with each other, similarly to excitations made simultaneously at different frequencies. Therefore, excitations made at different times may be combined as if they were taken simultaneously but at different frequencies.

When a plurality of intra-body electrodes is used, different electrodes may be injected with currents of different frequencies (unless the two electrodes share a differential excitation), e.g., in order to avoid interference between different excitations.

Combining data gathered at different times and at the same frequencies is similar to data gathered simultaneously at different frequencies. This is enabled due to the fact that the intra-body electrode carrier may be moved though the body lumen(s) thereby providing sets of spatial-related conductivity measurements representing large sets of conductance routes, which in turn enables enriching the 3D map of conductivity measurements and, as a result, improvement of the 3D resolution of the resulting image. According to embodiments of the present invention conductivity data (e.g., a collection of voltage measurements) may be collected at high rates, so the number of conductivity data sets that may be combined may be in the order of magnitude of hundreds, and even thousands, so as to enable producing data/images at a rate of, for example, 100 Hz.

The frequency difference may be sufficiently small so that frequency-independent conductivity may be assumed. For example, in some embodiments, the frequency difference between two frequencies that may be injected simultaneously is 0.1 kHz. The entire frequency change is up to 150% of the lowest frequency. Frequencies of between 10 kHz and 100 kHz may be used. It will be apparent that other center-frequencies and other ranges of frequency deviations may be used, for example in order to collect data related to conductivity of body tissues at other frequencies. According to some embodiments, for a frequency-independent conductivity according to a reasonable approximation, small frequency differences (for example, from 10 kHz to 15 kHz in 0.1 kHz jumps), may be used. For the collection of data also on conductivity at other frequencies, a 100 kHz frequency may be used, between 100 and 105 kHz.

Each electrode may measure voltages at each frequency, and it is assumed that the measurement is frequency independent. For example, if there are four intra-body electrodes, each of the intra-body electrodes is transmitting at a different frequency, and three pairs of surface electrodes, each pair transmitting at a different frequency, then each of the 10 electrodes may simultaneously measure voltages at 7 different frequencies.

When the catheter includes a plurality of electrodes, each time the catheter moves, the electrodes provide a different set of readings, and each such set may be reconstructed into a different conductivity image. The images can then be combined, e.g., by weighted averaging.

Alternatively, the readings may be combined into a single result set. For example, in the above-example of four intra-body electrodes and three pairs of surface electrodes, each position may provide seven readings at each of the 10 electrodes, resulting a total of seventy (70) readings. Accordingly, N positions of the catheter may provide 70N readings, that may be treated as if they were taken at 7N frequencies. Different frequencies that are used to imitate different positions do not interact with each other, so the total number of readings is 70N. This single data set of 70N readings may be processed to provide a single reconstruction of the imaged volume.

The conductivity distribution may be indicative of the anatomy, as different tissues have different conductivities, and even blood may have different conductivities depending on the oxygen concentration in it, so it is expected that, for example, the blood pool in the left atrium will show different conductivities at different distances from the ostium (openings) of the pulmonary veins. An anatomical image of a tissue (or a body organ) may be obtained from the conductivity distribution.

Reference is made now to FIG. 3 which schematically depicts electrical field generator/measurer 300, according to embodiments of the present invention. Field generator/measurer 300 of FIG. 3 depicts how two electrodes may be configured to transmit each at a different frequency, and receive (and measure) at this frequency, and at the frequency transmitted by the other electrode. Signal source 310 provides signal in frequency f1. This signal is fed to electrode, e.g., electrode 210 (of FIG. 2) via terminal point 350 and the signal reaches another electrode, e.g., electrode 212 (of FIG. 2) and received by it. Similarly, signal source 320 provides signal in frequency f2. This signal is fed to electrode 212 via terminal point 360 and the signal reaches electrode 210 and received by it. As a result, junction points 301 and 302 experience a multiplexed signal comprised of frequencies f1 and f2. D is a demultiplexer that is configured to receive, in the current example, multiplexed signal (comprising signals in frequencies f1 and f2) and enable only signal in one of the frequencies to pass through—signal in frequency f1 passes via D 332 and D 344 and signal in frequency f2 passes via D334 and D 342. Accordingly, voltmeter 312 measures the amplitude of the signal in frequency f1, as originated from signal source 310 and received by electrode 210, and voltmeter 314 measures the amplitude of signal in frequency f2 as originated from signal source 320 and received by electrode 210. The demultiplexing of the signals at section 300B of electrical field generator/measurer 300 is done in the same manner.

It will be apparent that for exciting more electrodes the sections 300A, 300B of electrical field generator/measurer 300 may be repeated. In some embodiments, other signal demultiplexers may be used, as is known in the art.

Reference is made to FIG. 4, which is a schematic block diagram of system 400 for conductivity-based imaging, according to embodiments of the present invention. System 400 may comprise main control unit 402 in active communication with surface electrodes unit 410 and intra-body electrodes unit 420, via communication channels 410A and 420A| respectively. Main control unit 402 may comprise controller 404 and signal generator/measurer 406, connectable via electrodes I/O interface unit 408. Control unit 402 may include a controller that maybe, for example, a central processing unit processor (CPU), a chip or any suitable computing or computational device, equipped with an operating system, a memory, an executable code, and a storage (not shown in order to not obscure the drawing). Main control unit 402 may be configured to carry out methods described herein, and/or to execute or act as the various modules, units, etc. More than one computing device may be included in a system according to embodiments of the invention, and one or more computing devices may act as the various components of the system. For example, by executing the executable code stored in the memory, the controller may be configured to carry out a method of acquiring signals from the electrodes for the construction of 3D imaging according to embodiments of the invention.

Signal generator/measurer 406 may produce signals in a manner similar to the description of the signals produced and measured by generator/measurer 300 of FIG. 3. Accordingly, signals may be fed to, and/or received from any of the body surface electrodes of surface electrodes unit 410 and intra-body electrodes, for example catheter electrodes, of intra-body electrodes unit 420. Body surface electrodes of unit 410 may be deployed and operated similarly to electrodes 102A/102B, 104A/104B 106A/106B of FIG. 1. Intra-body electrodes of unit 420 may be arranged and operable similar to electrodes 210, 212, 214 and 216 of FIG. 2.

Reference is made to FIG. 5, which is a top-level flow of process 500 for converting a collection of measured voltages on a set of electrodes into a 3D image, according to embodiments of the present invention. Plurality of electrical signals may be injected to the electrodes, surface electrodes and intra-body electrodes, for example catheter electrodes, according to one or more excitation schemes, as discussed above. A plurality of measured voltages v_{(i,j)} (502), measured at the plurality of electrodes, may be combined into a an averaged/weighted collection (or collections) V(i, j) (504) which then may be converted (or reconstructed) into large number of conductivity values σ_{(x, y, z)} (506), each is associated with a 3D point having a respective x, y, z spatial coordinates (508). The collection of spatial conductivity values may then be translated into a 3D image (510) that may be screened or otherwise presented.

Reference is made to FIG. 6A, which is a flow chart depicting method for conductivity-based imaging for imaging a body volume or for reconstructing body volume according to embodiments of the present invention. Body volume may include or be a body tissue. Currents may be injected, for example by control unit 402 using signal generator/measurer unit 406, to electrodes deployed on a patient's body, such as electrodes 410 of FIG. 4 (for example, electrodes 102A/B, 104A/B and 106A/B of FIG. 1), and to intra-body electrodes, for example catheter electrodes, such as electrodes 420 of FIG. 4, for example electrodes 210, 212, 214 and 216 of FIG. 2, according to an injection scheme (block 602). Injection scheme may include a time/frequency transmit scheme. Injection scheme may be controlled and monitored by controller 404. At block 604, voltages are measured on electrodes (e.g., on all electrodes) e.g. by signal generator/measurer 406, and an inverse problem (calculation and production of 3D imaging of conductance of body tissues based on the currents/voltages inverse calculations) (block 606) may be solved, e.g. by control unit 402, and a 3D conductance map (3D distribution of conductance measurements, also referred to herein as conductivity map) may be obtained and optionally provided for display (block 608). At block 610, a 3D image of the body tissue may optionally be produced (and optionally presented) based on the 3D conductance map.

The same general scheme is applied also according to some embodiments of the present invention, as depicted in FIG. 6B, to which reference is now made. FIG. 6B is a flow diagram depicting method for performing multiple cycles of measurements, according to the invention. According to some embodiments, 1^{st} set of electrodes are attached (or deployed) on a patient's body (block 652) and 2^{nd} set of electrodes are inserted in a patient's body lumen (block 654). Currents are injected according to a scheme (block 656), the scheme may include a time/frequency transmit scheme. Voltages are measured on electrodes (e.g., all electrodes) (block 658), and an inverse problem (calculation and production of 3D imaging of conductance of body tissues based on the currents/voltages inverse calculations) (block 660) is solved and a 3D conductance map (3D distribution of conductance measurements) is provided (block 662). At block 664, a 3D image of the body tissue may optionally be produced (and optionally presented) based on the 3D conductance map. It will be noted that according to some embodiments, the measurement of voltages developing of a set of surface and intra-body electrodes may rely on electrodes already deployed at the patient's body (e.g., otherwise deployed as part of a medical procedure). In such conditions, use may be made of existing electrodes and the steps of blocks 652, 654 may not be required.

Reference is made to FIG. 7, which is a flow chart depicting a method of performing conductivity-based imaging according to some embodiments of the present invention. This flow chart depicts a method of collecting and combining sets of spatial data points representing conductance between sets of electrodes. The example of FIG. 7 includes a defined number (M) of repetitions of data collection, beginning with setting the iteration numerator to 1 at block 702. The sets of electrodes are excited according to a given excitation scheme (block 704) and the voltage developing on the electrodes are measured and recorded (block 706). At the end of each cycle of excitation and voltage reading the value of the cycle numerator is checked (block 708). In case it is lower than M, another cycle is performed (blocks 704, 706). When the numerator has reached the value of M, the recorded measurements are combined (block 710) for enhanced resolution of the extracted 3D conductance image. The combined measurements are used for solving the inverse problem (block 712). The solved inverse problem is used to providing a 3D conductance map of the measured tissue (block 714) and a 3D image may optionally be produced (and optionally presented) based on the 3D conductance map (block 716). The repetitions that are described above are of measurements made with the intra-body electrodes at different locations. The readings may be most sensitive to the immediate surrounding of the electrode, so reading at different locations may provide information on the conductivity in many different locations. The repetitions may preferably relate measurements taken at different locations of the intra-body electrodes. The number of iterations may be determined solely by the measurements rate multiplied by the time of performing of the medical procedure. In some other or additional embodiments the number of repetition of measurements may be determined by other limiter(s).

FIG. 8A, to which reference is now made, is a simulation results of 3D conductivity values reconstructed from voltage measurements made by surface electrodes and in-body electrodes according to some embodiments of the invention. The simulation was performed using a cylinder of conductivity 1.0 that included two bodies, one of conductivity 1.5, and the other of conductivity 0 (the latter may imitate an air column). FIG. 8B shows the actual structure the reconstruction of which is depicted in FIG. 8A. Electrodes are attached to the surface of the cylinder to simulate the surface electrodes, and to the higher conductivity object, to simulate the in-body electrodes. The Maxwell equations were solved for the configuration of FIG. 8B for excitations of signals of common frequency and opposite phase from the surface electrode, and excitations of signals of different frequencies by each of the in-body electrodes. Voltage readings at each of the electrodes were obtained from that solution. These voltage readings were inputted to a solution of the inverse problem, under the boundary conditions that intra-body electrodes are inside the cylinder, and the surface electrodes are at the outer surface of the cylinder. The inverse problem was solved using the EIDORS software. As can be seen, the two objects are reconstructed with shape and conductivity values that are similar to those of FIG. 8B.

Various excitation schemes have been described. In one specific example of an excitation scheme, only one specific electrode (or only one pair or only one group of electrodes) has current injected to it at any given time and frequency and, as a result, transmits at that given time and frequency. Consequently, the transmissions from each transmitting electrode (or pair or group of transmitting electrodes) can be separated from the transmissions from other transmitting electrodes (or other pairs or groups of transmitting electrodes). At each time and frequency at which a transmitting electrode transmits, a resulting voltage may be measured by one or more receiving electrodes, so that for each transmitting electrode there is one or more pairs of electrodes including that transmitting electrode for the given time and frequency and a respective one of the receiving electrodes for that time and frequency. These pairs may comprise two surface electrodes, two in-body electrodes or one surface and one in-body electrode (respectively transmitting and receiving, or vice versa). Each pair of electrodes and the corresponding voltage measurement provide a data point that can then be used to solve the inverse problem of finding a conductivity map from the voltage measurements. In some specific cases, all transmitting electrodes may transmit at the same time and at different frequencies, at the same frequency but at different times or a combination of the two. It will be understood that at any given time, any one transmitting electrode may be a transmitting electrode for a given frequency and a receiving electrode at another frequency or frequencies, and/or may be a transmitting electrode for a given time slot or slots and a receiving electrode at another slot or slots. Any one receiving electrode may be a receiving electrode for a given frequency or frequencies and a transmitting electrode at another frequency or frequencies, and/or may be a receiving electrode for a given time slot or slots and a transmitting electrode at another slot or slots.

As mentioned above, position is not always needed to solve the inverse problem but electrode position can be used in some embodiments to find a conductance distribution as a solution to the inverse problem. For example, for each pair of excited electrodes (transmitting and receiving electrode), data indicative of a respective position is accessed for the transmitting electrode of the pair, that is the electrode of the pair to which current is applied, and for the receiving electrode of the pair, that is the electrode at which a voltage measurement is taken. The position is obtained in a suitable frame of reference, for example fixed on the body. In the case of surface electrodes, the position may be known based on a harness or vest containing the electrodes being placed on the body or by other optical or electromagnetic position measurements, for example using suitable markers. For the in-body electrodes, position may be obtained using a suitable imaging modality such as MRI or CT imaging, with suitable registration to a body frame of reference, or the positions may be obtained directly in a frame of reference defined by the surface electrodes by locating the in-body electrodes using navigation techniques based on the surface electrodes, as described above.

Reference is made in this application to solving an inverse problem or obtaining a solution to an inverse problem. It will be understood that this refers to inferring a spatial distribution of conductance or another dielectric property based on the collection of measurements V(i,j) discussed above, that is based on measurements of voltages generated on (surface and/or in-body) electrodes in response injection of currents to one or more (surface and/or in-body) electrodes. Finding the conductance distribution based on the measured voltages is the inverse of applying Maxwell or Laplace equations to currents applied at electrodes taking account of the conductance distribution and is hence referred to as solving an inverse problem. Many methods for doing this, numerical and otherwise will be known to the person skilled in the art and are available in commercially available software, for example as referenced above. One class of methods starts with a guess of a conductance distribution, applies the appropriate equations to the distribution and currents applied at one or more electrodes to produce predicted voltages at a set of electrodes at which voltages were measured. An error between the predicted and measured voltages is then used to adjust the conductance distribution and the process iterates until the error is reduced to a satisfactory level or another stopping criterion is met. One example of a class of methods of iteratively adjusting the conductance distribution in this way is gradient descent.

For the avoidance of doubt, some disclosed embodiments are set out in the following clauses:
1. A method of performing conductivity-based imaging comprising:
exciting at least one pair of electrodes according to an excitation scheme, the at least one pair of electrodes comprising a surface electrode located on the surface of an examined body and at least one in-body electrode located inside of the examined body;
measuring and recording voltages developing on the surface electrode and on the in-body electrode during the excitation according to the excitation scheme;
solving an inverse problem to achieve a 3D conductivity map from the recorded voltages; and
providing a 3D image of the body tissues based on the 3D conductivity map.
2. The method of clause 1, wherein the excitation is applied to at least one additional pair of electrodes that comprises a surface electrode located on the surface of the examined body and at least one in-body electrode located inside of the examined body.
3. The method of clause 1 or 2, wherein the excitation is applied to at least one additional pair of electrodes that comprises two in-body electrodes.
4. The method of any one of the preceding clauses, wherein the excitation is applied to at least one additional pair of electrodes that comprises two surface electrodes.
5. The method of any one of the preceding clauses, wherein the steps of exciting and measuring are repeated a defined number of times (M) with the in-body electrodes at different locations inside the body, wherein M is at least two.
6. The method of any one of the preceding clauses, wherein the steps of exciting and measuring are repeated at a rate of between 10 and 500 times per second.
7. The method of clause 5 or 6 further comprising a step of combining measurements obtained when the in-body electrodes were at different locations to a single set of measurements, and wherein the inverse problem is solved for that single set of measurements.
8. The method of clause 7, wherein the different locations include at least two locations, each in the vicinity of a different structural feature to be imaged within a volume of the examined body.
9. The method of clause 5, wherein the solving is executed for each of the M measurements separately, and the obtained solutions are averaged to provide the 3D image.
10. A method of providing a 3D image of the body tissues comprising:
exciting at least one pair of electrodes according to an excitation scheme, the at least one pair of electrodes comprising a surface electrode located on the surface of an examined body and at least one in-body electrode located inside of the examined body;
measuring voltages developing on the surface electrode and on the in-body electrode during the excitation according to the excitation scheme;
solving an inverse problem to achieve a 3D conductivity map from the measured voltages; and
providing a 3D image of the body tissues based on the 3D conductivity map.
11. The method of clause 10, wherein the excitation is applied to at least one additional pair of electrodes that comprises a surface electrode located on the surface of an examined body and at least one in-body electrode located inside of the examined body.
12. The method of clause 10 or 11, wherein the excitation is applied to at least one additional pair of electrodes that comprises two in-body electrodes.
13. The method of any one of clauses 10-12, wherein the excitation is applied to at least one additional pair of electrodes that comprises two surface electrodes.
14. The method of any one of clauses 10-13, wherein the steps of exciting and measuring are repeated a defined number of times (M) with the in-body electrodes at different locations inside the body, wherein M is at least two.
15. A method of obtaining a 3D conductivity map of body tissues comprising:
exciting at least one pair of electrodes according to an excitation scheme, the at least one pair of electrodes comprising a surface electrode located on the surface of an examined body and at least one in-body electrode located inside of the examined body;
measuring voltages developing on the surface electrode and on the in-body electrode during the excitation according to the excitation scheme; and
solving an inverse problem to obtain a 3D conductivity map from the measured voltages.
16. A method of performing conductivity-based imaging comprising:
receiving voltage measurements developed on a surface electrode located on the surface of an examined body and on an in-body electrode located inside of the examined body during an excitation according to an excitation scheme; wherein
the excitation scheme includes exciting the surface electrode and the in-body electrode;
solving an inverse problem to achieve a 3D conductivity map from the measured voltages; and
providing a 3D image of the body tissues based on the 3D conductivity map.
17. A system for performing conductivity-based imaging comprising a controller configured to perform the methods of clauses 1-16 when executing executable code stored in its memory.
18. A system for performing conductivity-based imaging comprising:
a control unit (402);
surface electrodes unit (410), comprising at least 2 electrodes;
intra-body electrodes (420), comprising at least 2 electrodes;
a first communication channel (410A) to provide communication between the control unit and the surface electrodes unit; and
a second communication channel (420A) to provide communication between the control unit and the intra-body electrodes unit.
19. The system of clause 18, wherein the control unit comprises a controller configured to perform the method of clauses 1-16 when executing executable code stored in its memory.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-37. (canceled)

38. A method of performing conductivity-based imaging comprising:
exciting at least one pair of electrodes according to an excitation scheme, at least one of the at least one pair of electrodes comprising a surface electrode located on the surface of an examined body and an in-body electrode located inside of the examined body;
measuring and recording voltages developing on the surface electrode and on the in-body electrode during the excitation according to the excitation scheme;
repeating the exciting, measuring, and recording with the in-body electrode or electrodes at different locations inside the body;
combining measurements obtained when the in-body electrode or electrodes were at different locations into a single set of measurements,
solving an inverse problem, for the single set of measurements, to obtain a 3D conductivity map from the recorded voltages; and
providing a 3D image of the body tissues based on the 3D conductivity map.

39. The method according to claim 38, wherein the excitation is applied to at least one additional pair of electrodes that comprises a surface electrode located on the surface of the examined body and an in-body electrode located inside of the examined body.

40. The method according to claim 38, wherein the excitation is applied to at least one additional pair of electrodes that comprises two in-body electrodes.

41. The method according to claim 38, wherein the excitation is applied to at least one additional pair of electrodes that comprises two surface electrodes.

42. The method according to claim 38, wherein the steps of exciting and measuring are repeated at a rate of between 10 and 500 times per second.

43. The method according to claim 38, wherein the different locations include at least two locations, each in the vicinity of a different structural feature to be imaged within a volume of the examined body.

44. The method according to claim 38, wherein the solving is executed for each of the M measurements separately, and the obtained solutions are averaged to provide the 3D image.

45. A method of obtaining a 3D conductivity map of body tissues comprising:
receiving measured voltages measured on receiving electrodes in response to currents injected to transmitting electrodes, the transmitting and receiving electrodes comprising at least one surface electrode located on the surface of an examined body and at least one in-body electrode located inside of the examined body; wherein the measured voltages comprise voltages measured with the at least one in-body electrode located in various locations inside the examined body, and
solving an inverse problem to obtain a 3D conductivity map from the received voltages.

46. The method according to claim 45, further comprising:
providing a 3D image of the body tissues based on the 3D conductivity map.

47. The method according to claim 45, wherein at least one of the receiving electrodes is a surface electrode.

48. The method according to claim 45, wherein at least one of the receiving electrodes is an in-body electrode.

49. The method according to claim 45, wherein at least one of the transmitting electrodes is a surface electrode.

50. The method according to claim 45, wherein at least one of the transmitting electrodes is an in-body electrode.

51. The method according to claim 45, wherein the measured voltages comprise one or more of a voltage measured on a surface electrode in response to current injected to an in-body electrode, a voltage measured on an in-body electrode in response to current injected to an in-body electrode, a voltage measured on an in-body electrode in response to current injected to a surface electrode.

52. The method according to claim 45, wherein measured voltages were measured at a rate of between 10 and 500 times per second.

53. The method according to claim 45, wherein the different locations include at least two locations, each in the vicinity of a different structural feature to be imaged within a volume of the examined body.

54. The method according to claim 45, wherein receiving the measured voltages composes receiving sets of measured voltages, wherein, for each set, respective measured voltages were measured on one or more of the receiving electrodes while injecting respective currents on one or more of the transmitting electrodes.

55. The method according to claim 54, wherein a measured voltage was measured on a selected electrode when obtaining one set of measurements and a current was injected to the selected electrode when obtaining a second set of measurements.

56. The method according to claim 45, wherein one or more of the transmitting electrodes are each associated with one or more other transmitting electrodes with a defined phase relationship between the respective currents of the associated electrodes.

57. The method according to claim 56, wherein one of the transmitting electrodes and another transmitting electrode are associated in a pair of transmitting electrodes having respective currents injected at the same frequency and opposite phases.

58. A system for obtaining a 3D conductivity map comprising a controller configured to perform a method according to claim 38, when executing executable code stored in its memory.
